(19) United States
(12) Patent Application Publication
(10) Pub. No.: US 2018/0137236 A1
(43) Pub. Date: May 17, 2018

(54) SYSTEM, METHOD AND DEVICE FOR IDENTIFYING DISCRIMINANT BIOLOGICAL FACTORS AND FOR CLASSIFYING PROTEOMIC PROFILES

(71) Applicants: Institut Pasteur de Montevideo, Montevideo (UY); Instituto Carlos Chagas, Fiocruz - Paraná, Curitiba (BR); Institut Pasteur, Paris (FR)

(72) Inventors: Paulo C. Carvalho, Curitiba (BR); Carlos Batthyány, Montevideo (UY); André R. F. Silva, Rio de Janeiro (BR); Diogo Borges Lima, Rio de Janeiro (BR); Valmir Carneiro Barbosa, Rio de Janeiro (BR); Alejandro Leyva, Montevideo (UY); Rosario Duran, Montevideo (UY); Julia Chamot-Rooke, Paris (FR)

(73) Assignees: Institut Pasteur de Montevideo, Montevideo (UY); Instituto Carlos Chagas, Fiocruz - Paraná, Curitiba (BR); Institut Pasteur, Paris (FR)

(21) Appl. No.: 15/814,788

(22) Filed: Nov. 16, 2017

Related U.S. Application Data
(60) Provisional application No. 62/422,964, filed on Nov. 16, 2016.

(51) Int. Cl. G06F 19/18; G06F 19/20; G01N 33/68

(52) U.S. Cl. CPC G06F 19/18; G01N 33/6803; G06F 19/20

(57) ABSTRACT

A system, method, computer readable medium and device for identifying discriminant spectrum clusters including receiving known input data set comprising spectra generated from biological samples known to either have or not have a biological condition where each spectrum may be either known to have been generated from the biological samples known to have or a biological condition, or from the biological samples known not to have same. A software module may apply quality control filters to the input data set to exclude spectra that do not meet the quality control filters, generate a set of remaining spectra, cluster same into a set of spectrum clusters by applying clustering parameters, and identify a set of discriminant spectrum clusters by examining whether each spectrum cluster exclusively contains only spectra generated from samples known to have a biological condition or exclusively contains spectra from samples known not to have the biological condition.

### CROSS REFERENCE TO RELATED APPLICATIONS

This application claims the benefit of U.S. Provisional Application No. 62/422,964, filed Nov. 16, 2016.

### BACKGROUND AND FIELD OF ART

The invention generally relates to the field of mass spectrometry, and of the analysis, evaluation and categorization of spectra generated through mass spectrometry.

The prevailing method for identifying discriminant biological factors is through peptide spectrum matching (PSM), which compares experimental spectra against those theoretically generated from a sequence database in order to attempt to identify a peptide (i.e., unknown spectra). However, a significant limitation of the current PSM method is that it cannot identify discriminant biological factors unless the sample matches the spectra of a known control, or reference data, sample.

This limitation stems, in part, from the conventional practice of analyzing biological factors as entire molecules. Thus, post-translational modifications (PTMs) or poor fragmentation of otherwise known biological factors will render them as unidentifiable. The implications of this limitation are significant. For example, when studying a disease such as cancer or comparing a resistant versus a non-resistant bacterial strain, there will be mutations or PTMs resulting from such an altered state. These modifications will ultimately be overlooked by typical proteomic pipelines.

Another widely adopted approach relies on obtaining a proteomic profile in a single spectrum and comparing it to those previously obtained and stored in a spectrum database. These approaches typically rely on growing a bacteria culture on a petri dish, enriching the sample for proteins (e.g., metal binding proteins), and obtaining a mass spectrum of the protein profile of this sample. A commercial example of this application is the MALDI Biotyper, from Bruker (https://www.bruker.com/products/mass-spectrometry-and-separations/maldi-biotyper/overview.html). Although this approach has proven effective, it fails when discriminating samples that are really close, such as, say, bacteria that is resistant or not to a drug.

This limitation stems, in part, given the complexity of the sample and trying to classify it within a single mass spectrum. Simply put, in many cases the discriminative factors are few and remain undetected by the experimental approach at hand.

Accordingly, the disclosed embodiments overcome the current limitations and enable the identification of discriminant spectrum clusters regardless of whether those spectra are within the database of known peptides or not. These clusters can originate from peptides, proteins (e.g., top down proteomics), or even metabolites (e.g., lipids).

### SUMMARY OF THE INVENTION

The following presents a simplified summary of the invention in order to provide a basic understanding of some aspects of the invention. This summary is not an extensive overview of the invention. It is intended to neither identify key nor critical elements of the invention nor delineate the scope of the invention. Its sole purpose is to present some concepts of the invention in a simplified form as a prelude to the more detailed description that is presented later.

The present invention is generally directed to a system, method, device and computer program product for categorizing significant biological conditions.

In some embodiments a system for identifying discriminant spectrum clusters may include a computer capable of receiving known input data set comprising a plurality of spectra generated from biological samples known to either have or not have a biological condition. Each spectrum in the known input data set may be either known to have been generated from the biological samples that are known to have or a biological condition, or known to have been generated from the biological samples that are known not to have a biological condition. The system may also include a software module that applies quality control filters to the known input data set to exclude spectra that do not meet the quality control filters and generate a set of remaining spectra. The software module may further cluster the remaining spectra into a set of spectrum clusters by applying clustering parameters, and identify a set of discriminant spectrum clusters by examining the spectrum clusters to identify for each spectrum cluster if it exclusively contains only spectra generated from samples known to have a biological condition or it exclusively contains spectra from samples known not to have the biological condition. The system may further include a display capable of displaying information about the discriminant the spectrum clusters.

In some embodiments a method for identifying discriminant spectrum clusters may include the steps of: (1) receiving known input data comprising a plurality of spectra generated from biological samples known to either have or not have a biological condition such that each spectrum in the known input data set may be either known to have been generated from the biological samples that are known to have or a biological condition, or known to have been generated from the biological samples that are known not to have a biological condition; (2) applying quality control filters to the known input data to remove spectra that do not meet the quality control filters and generate a set of remaining spectra; (3) clustering the remaining spectra into a set of spectrum clusters by applying clustering parameters; and (4) identifying a set of discriminant spectrum clusters by examining the spectrum clusters to identify for each spectrum cluster if it exclusively contains only spectra generated from samples known to have a biological condition or it exclusively contains spectra from samples known not to have the biological condition.

In some embodiments a computer readable medium containing program instructions for identifying discriminant spectrum clusters comprising, wherein execution of the program instructions by one or more processors of a computer system causes the one or more processors to carry out the steps of: (1) receiving known input data comprising a plurality of spectra generated from biological samples known to either have or not have a biological condition such that each spectrum in the known input data may be known to have been generated from samples that are known to have or to not have the biological condition; (2) applying quality control filters to the known input data to remove spectra that do not meet the quality control filters and generate a set of remaining spectra; (3) clustering the remaining spectra into a set of spectrum clusters by applying clustering parameters; and (4) identifying a set of discriminant spectrum clusters by examining the spectrum clusters to identify for each spectrum cluster if it exclusively contains only spectra generated from samples known to have a biological condition or it exclusively contains spectra from samples known not to have the biological condition.

In some embodiments a computing device for identifying biological factors may include input devices capable of receiving known input data set comprising a plurality of spectra generated from biological samples known to either have or not have a biological condition such that each spectrum in the known input data set may be either known to have been generated from the biological samples that are known to have or a biological condition, or known to have been generated from the biological samples that are known not to have a biological condition. The system may further include a software module that applies quality control filters to the known input data to remove spectra that do not meet the quality control filters and generate a set of remaining spectra. The software module may further cluster the remaining spectra into a set of spectrum clusters by applying clustering parameters, and identify a set of discriminant spectrum clusters by examining the spectrum clusters to identify for each spectrum cluster if it exclusively contains only spectra generated from samples known to have a biological condition or it exclusively contains spectra from samples known not to have the biological condition. The device may further include a display capable of displaying information about the discriminant the spectrum clusters.

In some embodiments a device for identifying discriminant spectrum clusters may include input devices capable of receiving known input data comprising a plurality of spectra generated from biological samples known to either have or not have a biological condition such that each spectrum in the known input data set may be either known to have been generated from the biological samples that are known to have or a biological condition, or known to have been generated from the biological samples that are known not to have a biological condition. The device may further include a software module that applies quality control filters to the known input data to remove spectra that do not meet the quality control filters and generate a set of remaining spectra. The software module may further cluster the remaining spectra into a set of spectrum clusters by applying clustering parameters. The software module may further identify a set of discriminant spectrum clusters by examining the spectrum clusters to identify for each spectrum cluster if it exclusively contains only spectra generated from samples known to have a biological condition or it exclusively contains spectra from samples known not to have the biological condition. They device may further include a display capable of displaying information about the discriminant the spectrum clusters.

### DESCRIPTION OF THE DRAWINGS

FIG. 1 illustrates an example of a suitable computing system environment on which features of the disclosed concept may be implemented.

FIG. 2 illustrates a workflow for a system in accordance with the disclosed concepts.

FIG. 3 illustrates a workflow for the knowledge base generation portion of a system in accordance with the disclosed concepts.

FIG. 4 illustrates a graphical user interface for controlling knowledge base generation in accordance with the disclosed concepts.

FIG. 5 illustrates a sample spectrum in accordance with the disclosed concepts.

FIG. 6 illustrates a sample spectrum and its cumulative curve.

FIG. 7 illustrates a sample spectrum and its cumulative curve.

FIG. 8 illustrates a sample graphical interface for discriminant cluster identification.

FIG. 9 illustrates a sample graphical user interface for spectrum cluster browsing.

FIG. 10 illustrates a sample graphical user interface for cluster distance representation.

### DETAILED DESCRIPTION OF ILLUSTRATIVE EMBODIMENTS

The present invention is generally directed to a system, method, device and computer program product for recording, analyzing and categorizing spectra generated from prepared biological samples to identify discriminant spectrum clusters, originating from biomolecules dissociated (or not dissociated) within a mass spectrometer that may be indicative of a biological condition. Accordingly, implementations of the invention include, or involve the use of computing devices.

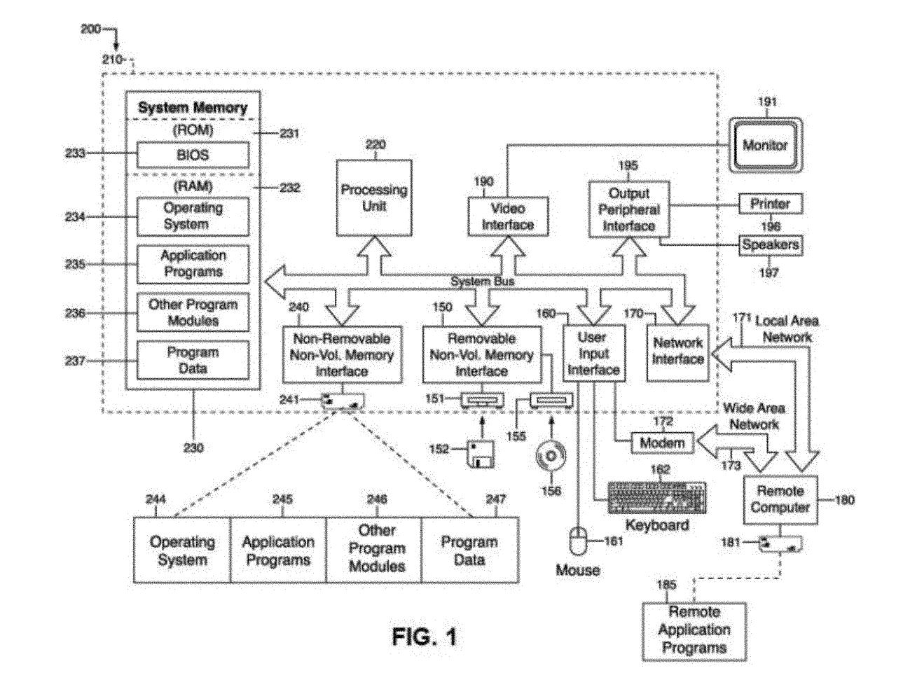

Specifically, embodiments of present invention may be implemented on one or more computing devices, including one or more servers, one or more client terminals, including computer terminals, a combination thereof, or on any of the myriad of computing devices currently known in the art, including without limitation, personal computers, laptops, notebooks, tablet computers, touch pads (such as the Apple iPad, SmartPad Android tablet, etc.), multi-touch devices, smart phones, personal digital assistants, other multi-function devices, stand-alone kiosks, etc. An exemplary computing device for implementing a computational device is illustrated in FIG. 1.

FIG. 1 illustrates an example of a suitable computing system environment 200 on which features of the invention may be implemented. The computing system environment 200 is only one example of a suitable computing environment and is not intended to suggest any limitation as to the scope of use or functionality of the invention. Neither should the computing environment 200 be interpreted as having any requirement relating to any one or combination of components illustrated in the exemplary operating environment 200.

The invention is operational with numerous other computing system environments or configurations. Examples of well-known computing systems, environments, and/or configurations that may be suitable for use with the invention include, but are not limited to, personal computers, server computers, hand-held, notebook or laptop devices, touch pads, multi-touch devices, smart phones, other multi-function devices, multiprocessor systems, microprocessor-based systems, set top boxes, programmable consumer electronics, network PCs, minicomputers, mainframe computers, distributed computing environments that include any of the above systems or devices, and the like.

The invention may be described in the general context of computer-executable instructions, such as program modules, being executed by one or more computing devices. Generally, program modules include routines, programs, objects, components, data structures, etc., that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices and internet or cloud-based storage devices.

With reference to FIG. 1, an exemplary system that may be used for implementing the invention includes a computing device 210 which may be used for implementing a client, server, mobile device or other suitable environment for the invention. Components of computing device 210 may include, but are not limited to, a processing unit 220, a system memory 230, and a system bus 221 that couples various system components including the system memory to the processing unit 220. The system bus 221 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computing device 210 typically includes a variety of computer readable media. Computer readable media may be defined as any available media that may be accessed by computing device 210 and includes both volatile and nonvolatile media, removable and non-removable media. By way of example, and not limitation, computer readable media may include computer storage media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 210. Combinations of the any of the above should also be included within the scope of computer readable media.

The system memory 230 may include computer storage media in the form of volatile and/or nonvolatile memory such as read only memory (ROM) 231 and random access memory (RAM) 232. A basic input/output system 233 (BIOS), containing the basic routines that help to transfer information between elements within computing device 210, such as during start-up, is typically stored in ROM 231. RAM 232 typically contains data and/or program modules that are immediately accessible to and/or presently being operated on by processing unit 220. By way of example, and not limitation, FIG. 1 illustrates operating system 234, application programs 235, other program modules 236, and program data 237.

The computing device 210 may also include other removable/non-removable, volatile/nonvolatile computer storage media. By way of example only, FIG. 1 illustrates a hard disk drive 240 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 241 is typically connected to the system bus 221 through a non-removable memory interface such as interface 240, and magnetic disk drive 151 and optical disk drive 155 are typically connected to the system bus 121 by a removable memory interface, such as interface 150.

The drives and their associated computer storage media discussed above and illustrated in FIG. 1, provide storage of computer readable instructions, data structures, program modules and other data for the computing device 210. In FIG. 1, for example, hard disk drive 241 is illustrated as storing operating system 244, application programs 245, other program modules 246, and program data 247. Note that these components can either be the same as or different from operating system 234, application programs 235, other program modules 236, and program data 237. Operating system 244, application programs 245, other program modules 246, and program data 247 are given different numbers here to illustrate that, at a minimum, they are different copies. A user may enter commands and information into the computer 20 through input devices such as a keyboard 162 and pointing device 161, commonly referred to as a mouse, trackball, touch screen, or multi-touch input device. Other input devices (not shown) may include a microphone, joystick, game pad, satellite dish, scanner, movement sensor device such as the Microsoft Kinect or the like. These and other input devices are often connected to the processing unit 220 through a user input interface 160 that is coupled to the system bus, but may be connected by other interface and bus structures, such as a parallel port, game port or a universal serial bus (USB). A monitor 191 or other type of display device may also be connected to the system bus 221 via an interface, such as a video interface 190. In addition to the monitor, computers may also include other peripheral output devices such as speakers 197 and printer 196, which may be connected through an output peripheral interface 195.

The computing device 210 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computing device 210, although only a memory storage device 181 has been illustrated in FIG. 1. The logical connections depicted in FIG. 1 include a local area network (LAN) 171 and a wide area network (WAN) 173, but may also include other networks. Such networking environments are commonplace in offices, enterprise-wide computer networks, intranets, the Internet, and cloud computing.

When used in a LAN networking environment, the computing device 210 is connected to the LAN 171 through a network interface or adapter 170. When used in a WAN networking environment, the computer 210 typically includes a modem 172 or other means for establishing communications over the WAN 173, such as the Internet. The modem 172, which may be internal or external, may be connected to the system bus 221 via the user input interface 160, or other appropriate mechanism. In a networked environment, program modules depicted relative to the computing device 210, or portions thereof, may be stored in the remote memory storage device. By way of example, and not limitation, FIG. 1 illustrates remote application programs 185 as residing on memory device 181. It will be appreciated that the network connections shown are exemplary and other means of establishing a communications link between the computers may be used.

In some embodiments a system for identifying discriminant spectrum clusters may include a computer capable of receiving known input data set comprising a plurality of spectra generated from biological samples known to either have or not have a biological condition. Each spectrum in the known input data set may be either known to have been generated from the biological samples that are known to have or a biological condition, or known to have been generated from the biological samples that are known not to have a biological condition. The system may also include a software module that applies quality control filters to the known input data set to exclude spectra that do not meet the quality control filters and generate a set of remaining spectra. The software module may further cluster the remaining spectra into a set of spectrum clusters by applying clustering parameters, and identify a set of discriminant spectrum clusters by examining the spectrum clusters to identify for each spectrum cluster if it exclusively contains only spectra generated from samples known to have a biological condition or it exclusively contains spectra from samples known not to have the biological condition. The system may further include a display capable of displaying information about the discriminant the spectrum clusters.

In some embodiments a method for identifying discriminant spectrum clusters may include the steps of: (1) receiving known input data comprising a plurality of spectra generated from biological samples known to either have or not have a biological condition such that each spectrum in the known input data set may be either known to have been generated from the biological samples that are known to have or a biological condition, or known to have been generated from the biological samples that are known not to have a biological condition; (2) applying quality control filters to the known input data to remove spectra that do not meet the quality control filters and generate a set of remaining spectra; (3) clustering the remaining spectra into a set of spectrum clusters by applying clustering parameters; and (4) identifying a set of discriminant spectrum clusters by examining the spectrum clusters to identify for each spectrum cluster if it exclusively contains only spectra generated from samples known to have a biological condition or it exclusively contains spectra from samples known not to have the biological condition.

In some embodiments a computer readable medium containing program instructions for identifying discriminant spectrum clusters comprising, wherein execution of the program instructions by one or more processors of a computer system causes the one or more processors to carry out the steps of: (1) receiving known input data comprising a plurality of spectra generated from biological samples known to either have or not have a biological condition such that each spectrum in the known input data may be known to have been generated from samples that are known to have or to not have the biological condition; (2) applying quality control filters to the known input data to remove spectra that do not meet the quality control filters and generate a set of remaining spectra; (3) clustering the remaining spectra into a set of spectrum clusters by applying clustering parameters; and (4) identifying a set of discriminant spectrum clusters by examining the spectrum clusters to identify for each spectrum cluster if it exclusively contains only spectra generated from samples known to have a biological condition or it exclusively contains spectra from samples known not to have the biological condition.

In some embodiments a computing device for identifying biological factors may include input devices capable of receiving known input data set comprising a plurality of spectra generated from biological samples known to either have or not have a biological condition such that each spectrum in the known input data set may be either known to have been generated from the biological samples that are known to have or a biological condition, or known to have been generated from the biological samples that are known not to have a biological condition. The system may further include a software module that applies quality control filters to the known input data to remove spectra that do not meet the quality control filters and generate a set of remaining spectra. The software module may further cluster the remaining spectra into a set of spectrum clusters by applying clustering parameters, and identify a set of discriminant spectrum clusters by examining the spectrum clusters to identify for each spectrum cluster if it exclusively contains only spectra generated from samples known to have a biological condition or it exclusively contains spectra from samples known not to have the biological condition. The device may further include a display capable of displaying information about the discriminant the spectrum clusters.

In some embodiments a device for identifying discriminant spectrum clusters may include input devices capable of receiving known input data comprising a plurality of spectra generated from biological samples known to either have or not have a biological condition such that each spectrum in the known input data set may be either known to have been generated from the biological samples that are known to have or a biological condition, or known to have been generated from the biological samples that are known not to have a biological condition. The device may further include a software module that applies quality control filters to the known input data to remove spectra that do not meet the quality control filters and generate a set of remaining spectra. The software module may further cluster the remaining spectra into a set of spectrum clusters by applying clustering parameters. The software module may further identify a set of discriminant spectrum clusters by examining the spectrum clusters to identify for each spectrum cluster if it exclusively contains only spectra generated from samples known to have a biological condition or it exclusively contains spectra from samples known not to have the biological condition. They device may further include a display capable of displaying information about the discriminant the spectrum clusters.

In certain embodiments, the quality control parameters may include a maximum Balance score threshold. In some embodiments, the maximum Balance score threshold may be set to 1.0. In some embodiments, the quality control parameters further include a minimum Xrea score. In some embodiments the minimum Xrea score may be set to 0.3.

In some embodiments, the clustering parameters may include a similarity threshold. In some embodiments, the similarity threshold may be set to 0.95. In some embodiments, a first spectrum may be clustered into a first spectrum cluster with a second spectrum if the dot product of a first normalized vector representing the first spectrum and a second normalized vector representing the second spectrum is greater than the similarity threshold. In some embodiments, a representative spectrum for the first spectrum cluster may be chosen based on the higher Xrea value between the first spectrum and the second spectrum. In some embodiments, the clustering parameters may include a retention time tolerance. In some embodiments, the retention time tolerance may be set to 10 minutes.

In some embodiments a principal component analysis (PCA) of the discriminant spectrum clusters may be generated.

Some embodiments may further involve receiving an unknown input data set comprising a plurality of spectra generated from other biological samples where it is unknown whether the other biological samples have the biological condition. In some embodiments, quality control filters may be applied to the unknown input data set to remove spectra that do not meet the quality control filters and generate a set of remaining unknown spectra. In some embodiments the remaining unknown spectra may be clustered into a second set of spectrum clusters by applying clustering parameters.

In some embodiments, the second set of spectrum clusters may be compared to the discriminant spectrum clusters. In some embodiments, the comparison of the second set of spectrum clusters to the set of discriminant spectrum clusters may be done by computing the Jaccard index of each cluster in the second set of spectrum cluster to each cluster in the set of discriminant spectrum clusters. Some embodiments may include identifying whether a biological condition is potentially present in a sample used to generate a spectrum in the second set of spectrum clusters based on the Jaccard index computed of at least one spectrum from the second set of spectrum clusters and at least one spectrum from the set of discriminant clusters. In some embodiments, the plurality of spectra in the known input data set further may be known to either to have been generated from the biological samples that are known to have or a second biological condition, or known to have been generated from the biological samples that are known not to have a second biological condition.

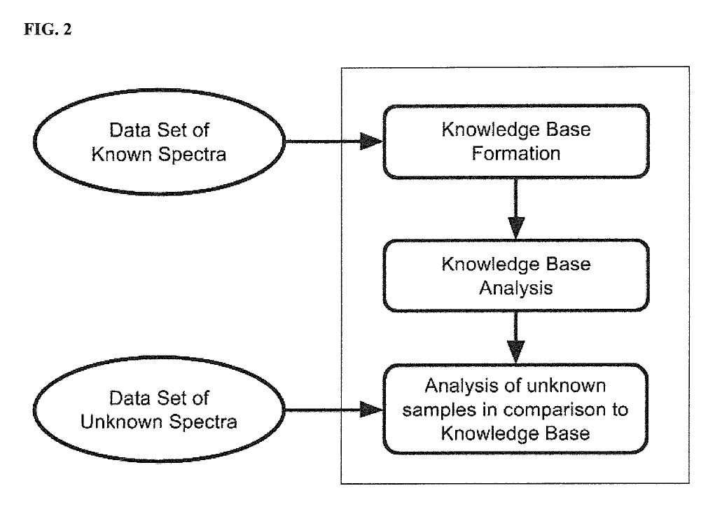

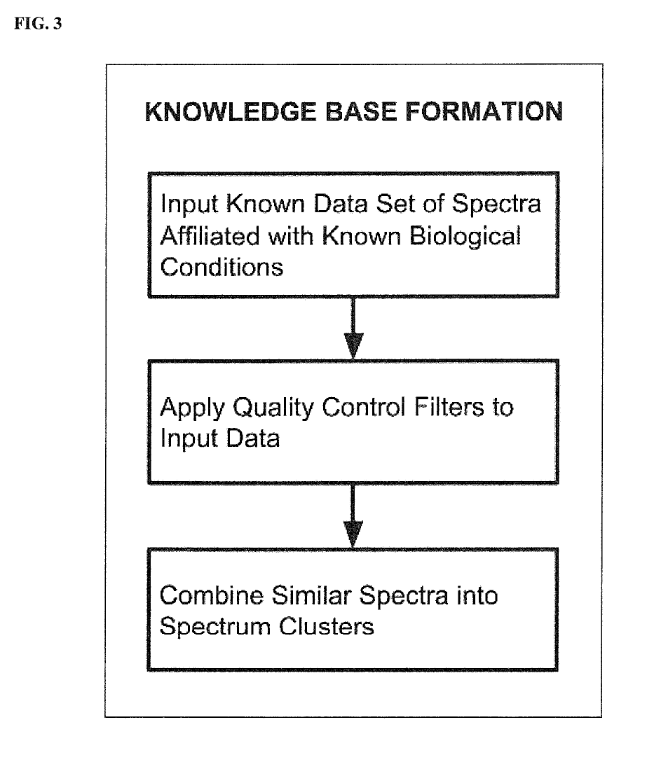

FIG. 2 depicts an exemplary flow diagram for a software system in accordance with the disclosed concepts. The system may include knowledge base formation functionality, knowledge base analysis functionality, and unknown sample analysis functionality. A flow diagram for the knowledge base formation functionality is depicted in FIG. 3. A knowledge base is formed from a known data set containing spectra generated from prepared biological samples for which a biological condition is known. For example, the known data set may be generated from spectra of prepared biological samples known to be the fungi Aspergillus flavus and Aspergillus oryzae. Alternatively, the data set may be formed from prepared biological samples from persons known to have a particular disease, and from persons known to not have that disease. A person of skill in the art will recognize that the disclosed concepts may be used to form a knowledge base from data relating to any biological conditions for which samples can be obtained that are known to have (or not have) that biological condition. The data set is formed by processing the samples known to have a certain biological condition via the methods described above. Accordingly, a set of spectra measurements that are affiliated with a biological condition is generated, and each spectrum corresponds to a biological factor—such as a peptide. The known database may include data representative of any number of biological conditions.

Generation of the data sets are derived from prepared biological samples analyzed by either MALDI-TOF-MS/MS or LC-ESI-MS/MS to generate a collection of raw dataset of tandem mass spectra (i.e., dissociated peptides, or even dissociated proteins, such as in the case of top-down experiments, or other biological material such as lipids). For example, the prepared biological samples A. flavus, A. oryzae, and A. parasiticus were analyzed using nano-chromatography coupled online with an Orbitrap Velos mass spectrometer according to protocols as described in Aquino, P. F. et al., “Are gastric cancer resection margin proteomic profiles more similar to those from controls or tumors?” J. Proteome Res. 11:5836-5842 (2012), which is incorporated by reference herein in its entirety.

Prepared biological samples for data set generation by mass spectrometry may involve multiple steps. In one embodiment, the biological sample may of a complex protein mixture that is first cleaved into peptides, either by chemical or enzymatic digestion, prior to MS analysis. The MS analysis is then performed on each of the individual peptides. Key steps in this strategy include the preparation of the protein sample for digestion, enrichment for any particular peptides of interest, and cleanup or desalting of the final peptide mixture prior to MS analysis by either MALDI-TOF-MS/MS (matrix-assisted laser desorption/ionization-time of flight tandem mass spectrometry) or LC-ESI-MS/MS (liquid chromatography-electrospray ionization tandem mass spectrometry). In another embodiment, the proteins need not to be broken down into peptides by digestion and may be analyzed as a whole. Proteins can be entirely dissociated in the mass spectrometer (e.g., top-down proteomics). Lipid samples may also be considered as an example of classifying biological conditions using metabolomic data. In other embodiments lipid samples may be used instead of proteins.

In one embodiment, the enzymatic digestion of a protein includes denaturing the biological factor by reducing disulfide bonds and alkylating free cysteines with dithiothreitol (DTT) and iodoacetamide (IAA). Following denaturing the biological factor digesting the denatured protein includes exposing the denatured protein with an enzyme solution that comprises proteases that break the peptide bonds holding the protein together. Typically, an enzyme solution will include trypsin and ammonium carbonate. However, depending on the complexity of the peptide mixture desired, different proteases may be chosen individually or sequentially. Some proteases include chymotrypsin, Lys-C, Asp-N, and Trypsin. Chemical processes for digesting proteins into peptides can also be used. One example chemical for protein digestion is cyanogen bromide in aqueous formic acid. Extraction of the peptides and sample cleanup, including desalting, subsequently follows before mass spectroscopy analysis.

Various methods and compounds for digesting proteins, such as those disclosed in Rebekah L. Gundry et al. “Preparation of Proteins and Peptides for Mass Spectrometry Analysis in a Bottom-Up Proteomics Workflow,” Curr Protoc Mol Biol, author manuscript: available in PMC 2010 Jul. 19, which is incorporated by reference herein in its entirety, are readily known to a person of ordinary skill in the art.

It will further be appreciated that the size of the data sets may grow quickly with the number of prepared biological samples. For example, running a single sample through the process described above may generate over 500,000 spectra. However, a known data set preferably has a sufficient number of samples to provide some statistical significance to the analysis performed. Accordingly, the known data may preferably have spectra generated from 30-40 samples, though the disclosed concepts may be practiced with data generated from, a lesser or greater number of samples.

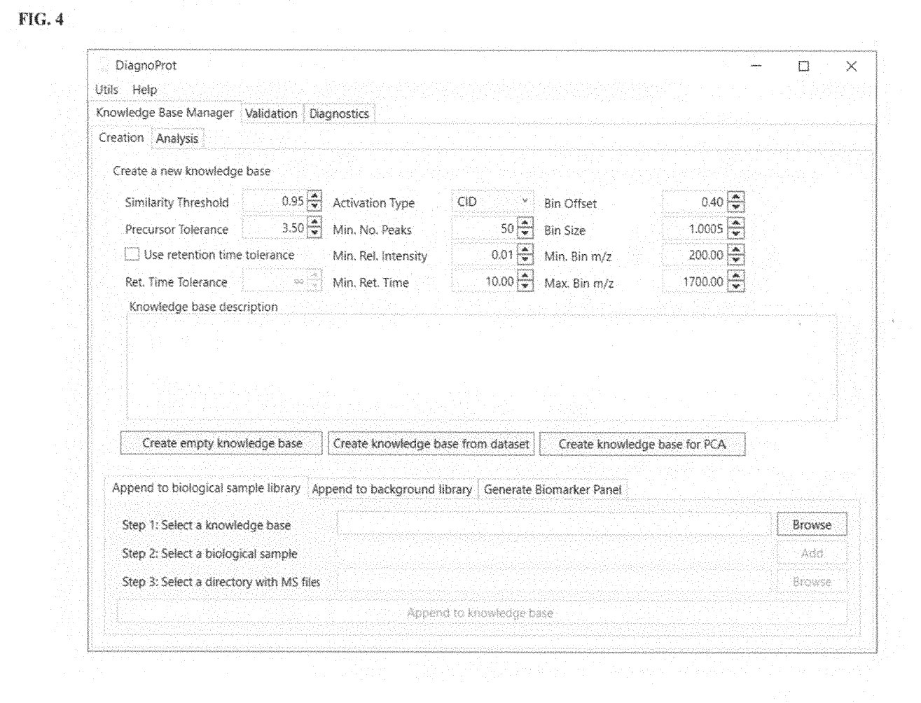

As shown in FIG. 3, the known data set is input into the system. The system may then apply quality control and data filters to the input to remove bad spectra data. Once the quality control filters have been applied, the system combines similar spectra into spectrum clusters for efficient analysis. FIG. 4 illustrates a graphical user interface which may be used to control the knowledge base formation process in accordance with the disclosed concepts. FIG. 4 depicts a number of data and quality control settings, thresholds and tolerances which manage the data input and processing. The settings for the data and quality control settings shown in FIG. 4 will necessarily depend on the processes and equipment used to generate the known data set. FIG. 4 shows preferred parameters and thresholds for standard laboratory and equipment settings using CID activation—for example most labs use trypsin to digest, reverse phase chromatography for 2 hours with acetonitrile going up to 40%, online with a mass spectrometer with an orbitrap instrument. Persons of skill in the art will recognize that the thresholds may vary as the equipment used, or the laboratory conditions change. Persons of skill in the art will also recognize that the disclosed concepts can be implemented with non-standard lab conditions, including but not limited to situations where other enzymes are used, where no digestion is performed, where lipids are analyzed, where different gradients of acetonitrile are applied, and where different equipment is used.

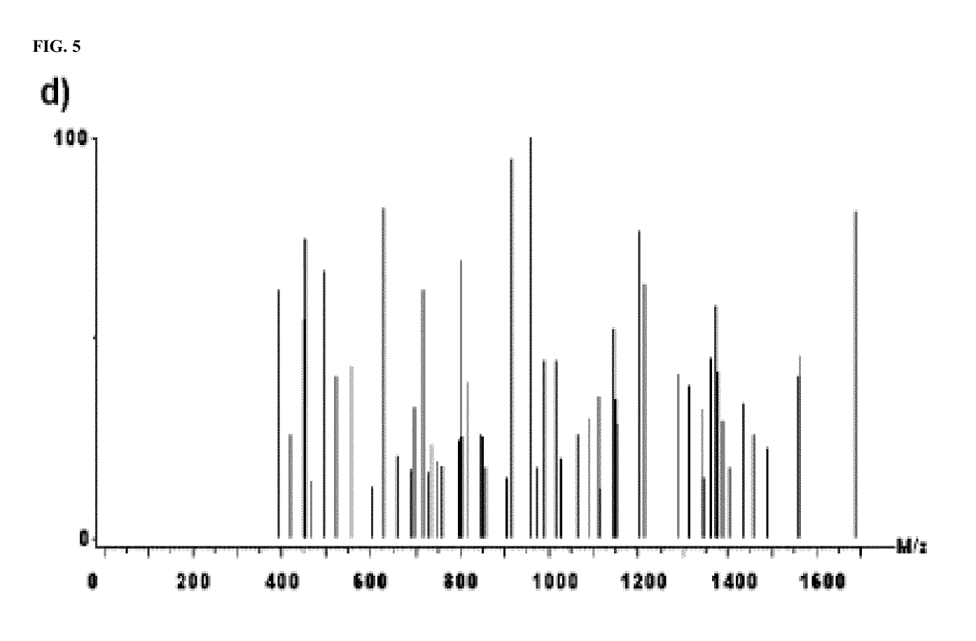

FIG. 5 illustrates a sample of a spectrum reading that may be input into the system as part of the known data set. A spectrum contains a set of measured intensities at various m/z values. Each spectrum in the known data set is either input into the system as a vector, or is converted into a vector upon being input into the system. The vectors formed from spectra may approximately contain approximately 2000 measurements. The size of the spectra input may vary depending on the resolution of the equipment used to generate same. Accordingly, the system may apply a binning algorithm to the spectra input according to a set of data parameters. Example data parameters are set forth in the right column of FIG. 4—Bin Offset, Bin Size, Min Bin m/z, Max Bin m/z.

As shown in FIG. 4, for example, data parameters may have a Min Bin m/z setting and a Max Bin m/z setting. Intensities measured at an m/z that is less than the Min. Bin m/z, or greater than the Max Bin m/z setting, may be disregarded or set to 0. Under standard lab settings, using ordinary lab equipment, the Min Bin m/z setting may be set to 200.00, and the Max Bin m/z setting may be set to 1700.00. These data filter values are chosen because 95% of intensity measurements fall within these m/z ranges. However, persons of skill in the art will recognize that practitioners using different equipment or lab settings, or analyzing samples or using enzymes that are expected to generate intensities at greater than 1700.00 m/z or less than 200.00 m/z may elect to change the Min & Max Bin m/z parameters to values that better conform to the data they are analyzing.

A binning procedure may also be applied to the spectra as they are input using a selected Bin Size and Bin Offset. As shown in FIG. 4 a Bin Size of 1.0005 may be used. This size is selected based on standard lab and equipment settings, in order to distinguish clearly between amino acids. As part of the binning process, multiple intensities measured within 1.0005 m/z values of each other may be combined. This size is chosen to distinguish between different amino acids in peptides under standard laboratory conditions. As shown in FIG. 4, a bin offset may also be applied in order to help distinguish between amino acids and combinations of amino acids. The bin offset is preferably set to 0.40, because no combination of sums of amino acid mass will coincide with the sum of the offset plus a multiple of the bin number. As discussed above, the ideal data parameters used will vary depending on the equipment used. When using state-of-the-art high resolution equipment, no offset may be necessary.

As shown in the middle column of FIG. 4, numerous quality control factors may be used to further restrict the data that is accepted into the knowledge base. For example, unknown biomolecules (e.g., peptides) for a biological condition (e.g., containing a mutations or post-translational modification) may not ionize well in the mass spectrometer, and that should be kept out of the knowledge base as their spectra would not contribute with discriminative information. One such quality control parameter is the minimum number of peaks, shown as Min No. Peaks in FIG. 4. It is set to 50 by default. This threshold has been empirically determined based on the data generated by the method described above under standard laboratory conditions. Molecules that do not break up correctly in the mass spectrometer have a small number of intensity peaks, and may be disregarded.

Similarly, a minimum relative intensity parameter may be used, such as a minimum relative intensity filter parameter, shown as Min. Rel. Intensity in FIG. 4. Applying this control, intensities measured at less than 0.01 times the highest intensity measured in a spectrum are disregarded when calculating whether the minimum number of peaks parameter is met. Accordingly, where the measured intensity of a peak divided by the maximum intensity in the spectrum is less than minimum relative intensity parameter, the peak may be discarded. The vector data for these measurements may also be zeroed. Again, a person of ordinary skill in the art will recognize that depending on what is being measured, and the laboratory equipment and conditions used, the desired threshold may vary.

A minimum retention time may also be used to filter data, shown as Min. Ret. Time in FIG. 4. It may be preferable to set this parameter to 10.00 minutes. Spectra generated before the value set in this parameter are disregarded. The appropriate setting for this parameter, also depends on the equipment, laboratory conditions and samples being processed. For example, if lipids are being processed under standard laboratory settings, a parameter setting of 20.00 minutes may be preferable. Similarly, the acetonitrile-to-water gradient used in the mass spectrometer may also affect this parameter. For example, a setting of 10.00 minutes may be appropriate for a process going from 5% to 40% acetonitrile over two hours. If the process applies a gradient of 5% to 40% acetonitrile over one hour, a 5.00 minutes minimum retention time may be more appropriate. The motivation is that peptides will, in most cases, only elute from the chromatographic column after a certain percentage of ACN is achieved.

Quality control calculations may also be considered in determining whether a spectrum should be included in the knowledge base or not, including the Xrea and Balance calculations described below.

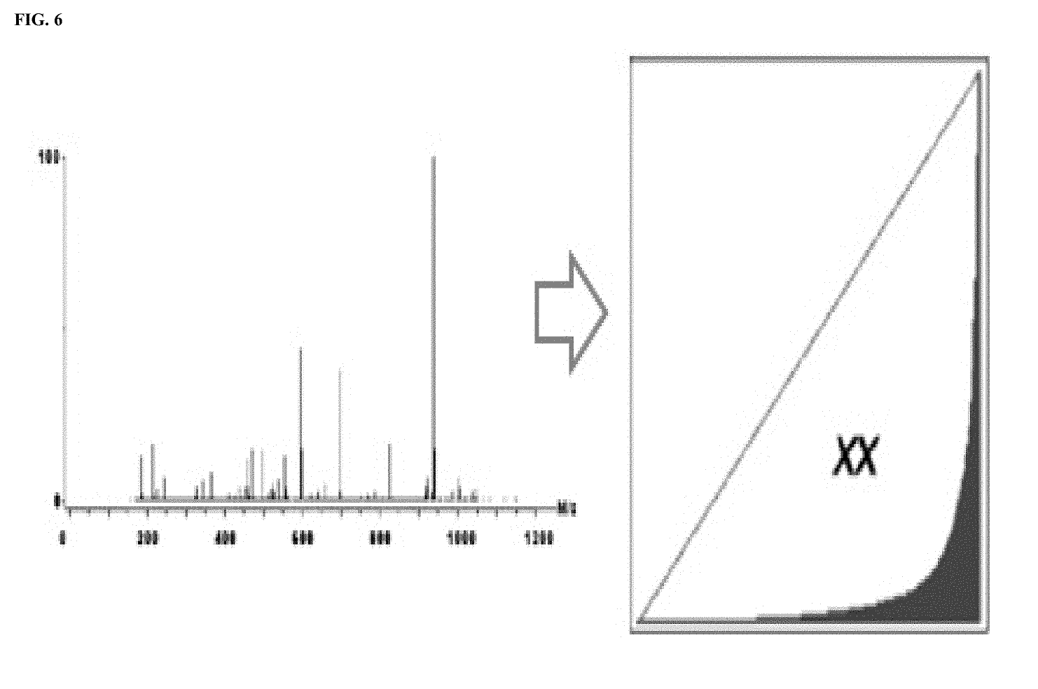

The Xrea calculation is a signal-to-noise ratio calculation described by Na and Paek in their 2006 paper, “Quality Assessment of Tandem Mass Spectra Based on Cumulative Intensity Normalization,” which is incorporated by reference herein in its entirety. As Shown in FIG. 6, the Xrea of a spectrum is calculated as follows:

$$Xrea = \frac{Area XX}{Area of triangle + α}$$

Area XX is the area of the triangle, less the area of the cumulative curve. The cumulative curve is formed by lining a spectrum's peaks in ascending order of cumulative normalized intensity. The cumulative normalized intensity of the for the nth highest peak is calculated as follows:

$$C N I ( n ) = \frac{\sum \{ Iraw ( x ) \vert Rank ( x ) \geq n \}}{TIC}$$

I_{raw}(x) is the raw intensity measured at x (m/z), Rank(x) is the order is the index of peak x where the peaks are ordered in descending order, and TIC is the total raw intensity of all peaks in the spectrum. Accordingly, the cumulative normalized intensity of the highest peak, CNI(1), is 1, because it is the sum of the raw intensities of all peaks divided by same. Similarly, the cumulative normalized intensity of the second highest peak, CNI(2), is TIC less the raw intensity of the highest peak divided by TIC. The difference between CNI(n) and CNI(n−1) is defined as nth RI_{by TIC}

The area of cumulative curve is computed using strip method of numerical integration. Bin width is fixed as 1/n, where n is the number of fragment ion peaks. Penalty factor, α, is defined as the relative magnitude of the most abundant peak. Thus a is the most intense RI_{by TIC}, as defined above.

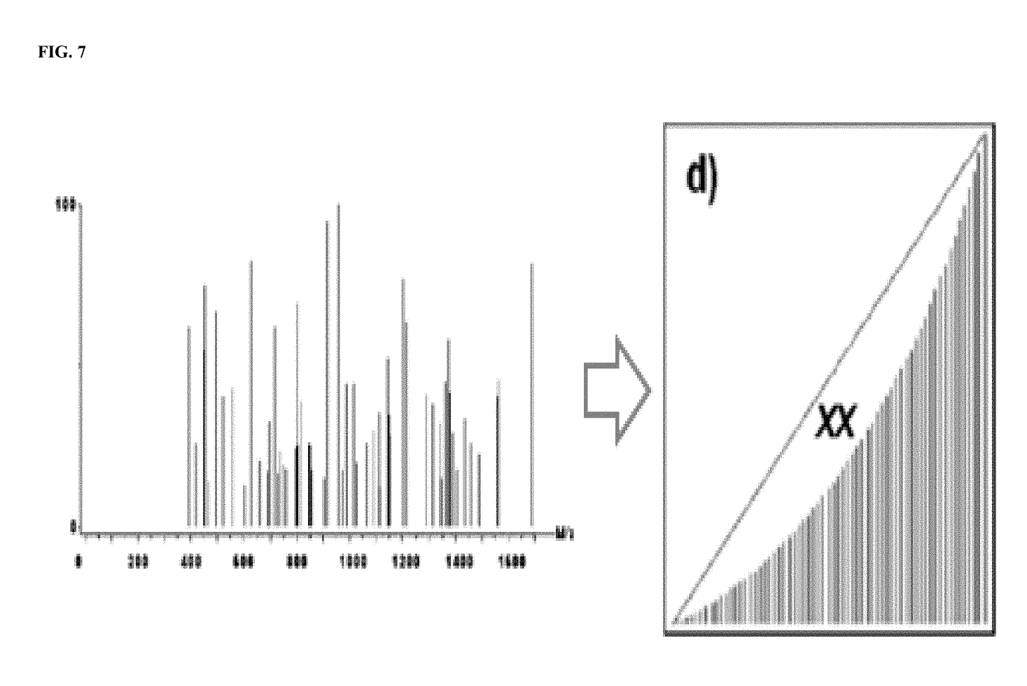

The more intense the magnitude of the most abundant peak is, the larger the area of XX, and thus, the spectrum will be regarded as having better quality. The penalty factor is employed to balance this, and its value is the most intense RI_{by TIC} in each spectrum. FIGS. 6 & 7 show cumulative curves for their respective spectra. The curve in FIG. 6 generates the higher Xrea, and is therefore considered a higher quality spectrum than that in FIG. 7. An Xrea threshold may be used to determine which spectra should be kept and which should be discarded. It may be advantageous to use an Xrea threshold of 0.4, such that spectra having Xrea values of less than 0.4 are discarded. Persons of skill in the art will recognize that the Xrea threshold may be varied depending upon the amount of culling of spectra desired—the higher the threshold, the fewer the spectra that will meet same.

The system may also use a Balance score for quality control. The Balance score measures the difference between the average peak distribution of a reference data set, and the peak distribution of a given spectrum from the known data set. A probability density function G_{z} is estimated from the intensity distribution of the reference data set. The spectra are first binned into 100 m/z bins, aggregating the values of all peaks within each 100 m/z. Then, an average spectrum is obtained from this reference set of binned spectra, and the binned intensities are normalized so that the sum of the intensities equals 1. To calculate balance of spectrum m, a discrete probability distribution B(m) is obtained using the same binning procedure on m and normalizing the intensities of B (m) so that the sum equals 1. Balance is calculated using the Kullback-Leibler divergence from the normalized intensities of the probability distribution B(m) with the probability distribution G_{z} expected from known spectrum from a reference collection, such as a reference distribution from a dataset from a Library of Model Organisms.

Balance(m)=D_{KL}(B(m)∥G_{z})

Kullback-Leibler divergence is well known in the art, and calculates the divergence of two probability density functions as the sum of all values of the first probability function times the natural log of the first probability density function divided by the second function for each possible value of the two probability functions.

$${D}_{KL} ( P \vert \vert Q ) = {\sum }_{i} P ( i ) ln ( \frac{P ( i )}{Q ( i )} )$$

Accordingly, the Balance measure compares the recorded spectra from the known data set to the expected spectra from reference data identified with high confidence. It may be advantageous to use a Balance threshold to discard spectra having high Balance scores. It may further be advantageous to use a Balance threshold of 1.0. Persons of skill in the art will recognize that the Balance threshold may be varied depending upon the amount of culling of spectra desired—the lower the threshold, the fewer the spectra that will meet same.

The clustering process may be governed by a set of clustering parameters as shown in the left column of FIG. 4. These may include parameters for precursor tolerance, and retention time tolerance. A similarity threshold may also be considered. Once the quality control settings have been run, the spectra input into the system are clustered such that one representative cluster is chosen to represent a cluster of similar vectors. Spectra are clustered using a Similarity Threshold. As shown in FIG. 4, a similarity threshold of 0.95. Again, persons of ordinary skill in the art will recognize that the Similarity Threshold chosen may vary based on the resolution of the equipment used and the data being analyzed. Where state-of-the-art, high resolution equipment is being used, or where smaller clusters are desired for analysis, a higher threshold may be used. Where lower resolution equipment is used, or larger clusters are desired, a lower threshold may be used. Clusters may be formed such that they are restricted to having spectrum that are only from a biological condition. Alternatively, clusters may be formed to include spectrums associated with any biological condition in the known data set.

The precursor tolerance considers the mass of the original molecule when combining spectra. In FIG. 4, the precursor tolerance is set to 3.50. Accordingly, if the absolute differences in m/z units of the original molecule is greater than 3.5 m/z between two spectra considered for clustering, then these spectra should not be clustered. As before, the quality of equipment used may affect the ideal tolerance to be used.

Retention time tolerance may be used to establish a maximum amount of time between when spectra are measured such that if exceeded the spectra should not be clustered. As shown in FIG. 4, this is set to 20 minutes. Alternatively, in some implementations it may be set to 10 minutes. Ultimately the appropriate measure for this parameter will depend on the equipment and laboratory conditions being used. Alternatively, the user may leave this value unset, or disabled, such that the difference in time between when spectra are measured is not considered during the clustering process.

The clustering process may also be governed by the binned base peak comparison. The binned base peak of a binned spectrum is defined as the bin with the highest sum of intensities. If the binned base peaks of the spectra are not the same, then the spectra should not be clustered.

If the tolerances parameters do not prohibit clustering and if the binned base peaks of the spectra are the same, the vectors representing the spectra in the known data set may be compared for similarity and clustered. As part of this, the vectors may be normalized to have a magnitude of one (1). A measure of similarity, such as, the dot product, of each vector is then calculated with other vectors. Persons of ordinary skill in the art will recognize that other suitable similarity measures known in the art, or to be developed in the future, may be used in accordance with the disclosed concepts. Another example of metric of similarity can be the cross-correlation. Where similarity of two vectors exceeds the similarity threshold, the vectors are clustered. A representative vector may be selected by keeping the spectrum having the highest Xrea score, by simply keeping the first vector in the cluster as representative, or by using any other heuristic that may be applied in order to select the representative vector. For example, the representative vector for each cluster may be selected based on the vector that maximizes the sum of its dot products with other vectors in the cluster. Persons of skill in the art will recognize that many different algorithms that are known in the art or that will be developed can be applied to selecting representative vectors for each cluster in accordance with the disclosed concepts.

Once clustering has completed the system may finalize the knowledge base. In doing so the system may store a condition collection comprising all clusters affiliated with a condition, and may store each such collection into a knowledge base file. Alternatively, the cluster may store all of the clusters together, and further store a condition data set, which may include an array or a list of all clusters pertaining to that biological condition. This data may be stored in any manner known in the art, locally, or on networked or cloud servers.

The system may examine clusters to identify whether these clusters are discriminative. Specifically, for each pair of biological conditions, A and B, the system may examine each cluster and determine whether it has members in that pertain to only condition A, to only condition B, to both conditions A and B, or to neither condition (for data sets involving more than two biological conditions). Where the members of clusters pertain only to one, and not to both, biological conditions, that cluster may be identified as a discriminant spectrum cluster corresponding to a discriminant biological factor. In implementations where clusters are restricted to having members associated with only one biological condition, the determination of whether such clusters are discriminant or not can be made by applying a similarity threshold between each such cluster, and each of the clusters associated with other biological condition. In other words, for each cluster that pertains to condition A, we check the similarity of that cluster against each cluster that pertains to condition B. If the similarity of the cluster pertaining to condition A is similar to a cluster that pertains to condition B (i.e. if the dot product of the vectors of the representative spectrum exceeds the similarity threshold, or any other suitable similarity metric), then the cluster can be said to be shared between conditions A and B. However, if the cluster pertaining to condition A is not similar to any cluster pertaining to condition B, then the cluster pertaining to condition A can be identified as a discriminant cluster for condition A. The same process can then be done to identify discriminant clusters for condition B, and for any other conditions being considered.

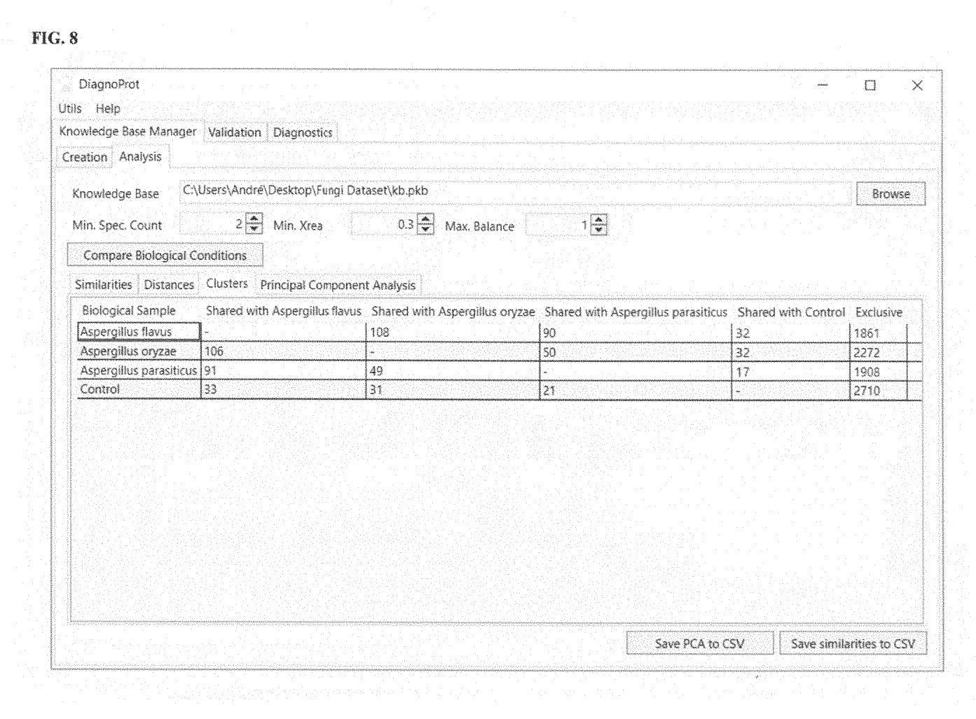

The system may then display a table listing how many discriminant clusters exist. A sample table interface is illustrated in FIG. 8. As shown in FIG. 8, the table may display a count of how many spectrum clusters are affiliated with multiple biological conditions, as well as a count of how many clusters are exclusive to each biological condition. This table interface may also allow a user to specify a minimum spectrum parameter, a minimum Xrea parameter or a maximum Balance parameter. These controls filter out clusters having less than the selected minimum number of spectra, clusters having an Xrea score less than the minimum Xrea, or clusters having Balance score more than the maximum balance score, respectively.

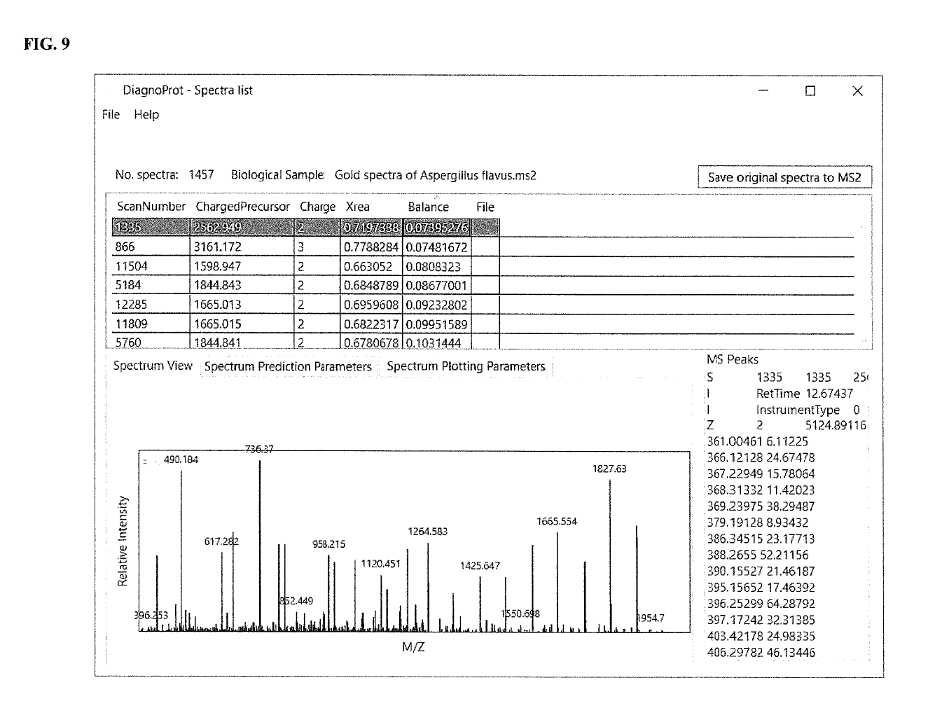

The system may allow a user to click on a cell containing such a count of cluster nodes, and open a spectrum cluster browser. An example spectrum cluster browser is illustrated in FIG. 9, which enables viewing each member spectrum in that cluster together with detailed information (e.g., charge state, precursor m/z, etc.) and QC scores such as Xrea and Balance.

The spectrum cluster browser may also provide the capability to running a search, for example using the Comet search engine, in an attempt to identify (i.e., assign a peptide sequence) to the spectra in that cluster. The spectrum cluster browser may also allow the user to calculate and add XCorr values in addition to the existing Xrea and Balance scores. This enables shortlisting spectrum clusters having good Xrea and Balance scores that nevertheless remained unidentified by Comet (because, e.g., of having XCorr <1.5). These discriminant spectrum clusters, are exclusive to their respective biological condition and therefore may correspond to discriminant biological factors. However, they remain unidentified by standard proteomic identification procedures. Accordingly, they qualify for further examination with complementary experimental methods, computational proteomic identification algorithms or other research efforts to identify the underlying biological factor.

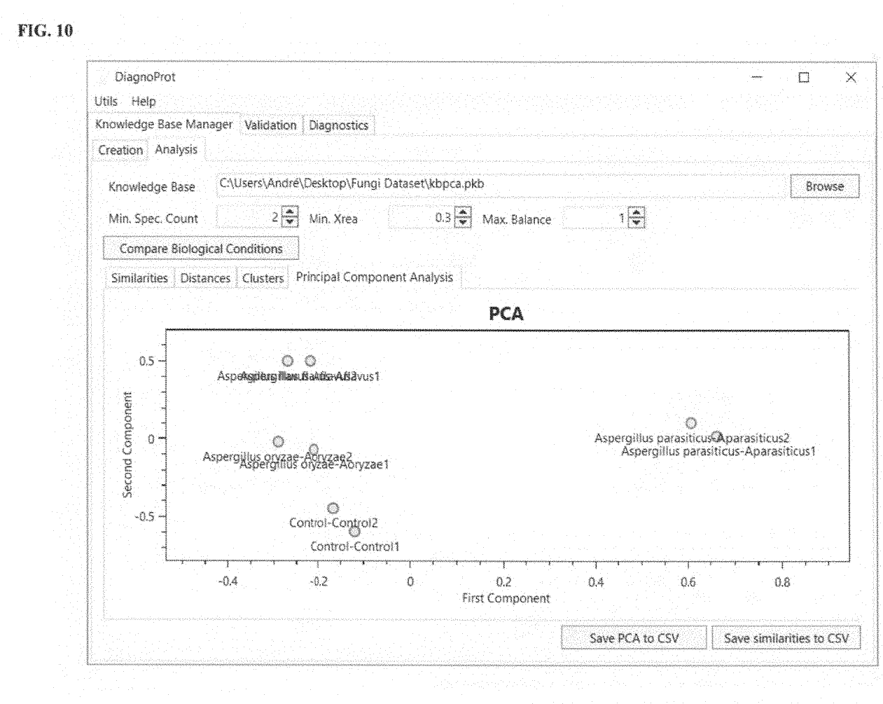

The system may further enables generating a principal component analysis (PCA), or make use of other types of multidimensional scaling strategies, to plot each biological assessment (i.e., each mass spectrometry analysis) included in its knowledge base. This is useful in that it may provide a bird's-eye view as to how “proteomically close” two biological conditions are to each other. For example, a PCA plot for the Aspergillus dataset is given in FIG. 10. Such a plot is obtained from the square matrix comprising the Jaccard indices between the spectral collections of each pair of biological replicates in the knowledge base. Specifically, the Jaccard index between every two collections of spectral nodes, generated from each mass spectrometry analysis, is computed. The Jaccard index between two sets A and B is defined as

|A∩B|/|A∪B|

Once all indices are at hand, a dimensionality reduction (from the total number of replicates to 2) is achieved via PCA. The resulting plot shows that samples from the same condition cluster together naturally. The PCA interface may also allow a user to specify a minimum spectrum parameter, a minimum Xrea parameter or a maximum Balance parameter. These controls filter out from considerations clusters having less than the selected minimum number of spectra, clusters having an Xrea score less than the minimum Xrea, or clusters having Balance score more than the maximum balance score, respectively.

The system may also receive subsequent input of an unknown data set comprising spectra generated from unknown samples—where it is unknown if the samples (or the spectra) have a biological condition. The spectra from these samples may be analyzed as discussed above, applying quality control parameters and clustering. The spectra may be further analyzed to identify whether any clusters generated from same match the discriminant clusters present in the knowledge base. Where a close match exists, the unknown sample may be classified as potentially indicative of the biological conditions corresponding to the discriminant spectrum cluster from the knowledge base to which it is closest. This may be achieved by applying the same quality control procedures, clustering all corresponding spectra, and then computing the Jaccard index between each query cluster and the cluster of each biological condition in the knowledge base. Persons of ordinary skill in the art will recognize that the Jaccard index is one of many comparisons that may be used to determine the closeness of the unknown spectra to the spectra in the knowledge base within the scope of the disclosed concepts. Any such comparative functions known in the art may be implemented. Of the latter, the condition yielding the highest Jaccard index can be flagged as the most likely assignment to the query cluster in question. Alternatively, the spectra, or clusters, from the unknown sample may be considered using the clustering parameters discussed above to determine whether they would qualify for clustering with the existing discriminant clusters in the knowledge base, which would yield additional confidence that the clusters from the unknown sample may be indicative of the presence of the biological condition relating to such clusters from the knowledge base.

I claim:

1. A system for identifying discriminant spectrum clusters comprising:
a computer capable of receiving known input data set comprising a plurality of spectra generated from biological samples known to either have or not have a biological condition such that each spectrum in the known input data set is either known to have been generated from the biological samples that are known to have or a biological condition, or known to have been generated from the biological samples that are known not to have a biological condition;
a software module that
applies quality control filters to the known input data set to exclude spectra that do not meet the quality control filters and generate a set of remaining spectra;
clusters the remaining spectra into a set of spectrum clusters by applying clustering parameters; and
identifies a set of discriminant spectrum clusters by examining the spectrum clusters to identify for each spectrum cluster if it exclusively contains only spectra generated from samples known to have a biological condition or it exclusively contains spectra from samples known not to have the biological condition; and
a display capable of displaying information about the discriminant the spectrum clusters.

2. A method identifying discriminant spectrum clusters comprising the steps of:
receiving known input data comprising a plurality of spectra generated from biological samples known to either have or not have a biological condition such that each spectrum in the known input data set is either known to have been generated from the biological samples that are known to have or a biological condition, or known to have been generated from the biological samples that are known not to have a biological condition;
applying quality control filters to the known input data to remove spectra that do not meet the quality control filters and generate a set of remaining spectra;
clustering the remaining spectra into a set of spectrum clusters by applying clustering parameters; and
identifying a set of discriminant spectrum clusters by examining the spectrum clusters to identify for each spectrum cluster if it exclusively contains only spectra generated from samples known to have a biological condition or it exclusively contains spectra from samples known not to have the biological condition.

3. A computer readable medium containing program instructions for identifying discriminant spectrum clusters comprising, wherein execution of the program instructions by one or more processors of a computer system causes the one or more processors to carry out the steps of:
receiving known input data comprising a plurality of spectra generated from biological samples known to either have or not have a biological condition such that each spectrum in the known input data is known to have been generated from samples that are known to have or to not have the biological condition;
applying quality control filters to the known input data to remove spectra that do not meet the quality control filters and generate a set of remaining spectra;
clustering the remaining spectra into a set of spectrum clusters by applying clustering parameters; and
identifying a set of discriminant spectrum clusters by examining the spectrum clusters to identify for each spectrum cluster if it exclusively contains only spectra generated from samples known to have a biological condition or it exclusively contains spectra from samples known not to have the biological condition.

4. A computing device for identifying biological factors comprising:
input devices capable of receiving known input data set comprising a plurality of spectra generated from biological samples known to either have or not have a biological condition such that each spectrum in the known input data set is either known to have been generated from the biological samples that are known to have or a biological condition, or known to have been generated from the biological samples that are known not to have a biological condition;
a software module that
applies quality control filters to the known input data to remove spectra that do not meet the quality control filters and generate a set of remaining spectra;
clusters the remaining spectra into a set of spectrum clusters by applying clustering parameters;
identifies a set of discriminant spectrum clusters by examining the spectrum clusters to identify for each spectrum cluster if it exclusively contains only spectra generated from samples known to have a biological condition or it exclusively contains spectra from samples known not to have the biological condition; and
a display capable of displaying information about the discriminant the spectrum clusters.

5. A device for identifying discriminant spectrum clusters comprising:
input devices capable of receiving known input data comprising a plurality of spectra generated from biological samples known to either have or not have a biological condition such that each spectrum in the known input data set is either known to have been generated from the biological samples that are known to have or a biological condition, or known to have been generated from the biological samples that are known not to have a biological condition;
a software module that
applies quality control filters to the known input data to remove spectra that do not meet the quality control filters and generate a set of remaining spectra;
clusters the remaining spectra into a set of spectrum clusters by applying clustering parameters;
identifies a set of discriminant spectrum clusters by examining the spectrum clusters to identify for each spectrum cluster if it exclusively contains only spectra generated from samples known to have a biological condition or it exclusively contains spectra from samples known not to have the biological condition;
a display capable of displaying information about the discriminant the spectrum clusters.

6. The invention of claims 1-5 wherein the quality control parameters comprise a maximum Balance score threshold.

7. The invention of claim 6 wherein the maximum Balance score threshold is set to 1.0.

8. The invention of claim 7 wherein quality control parameters further comprise a minimum Xrea score.

9. The invention of claim 8, wherein the minimum Xrea score is set to 0.3.

10. The invention of claims 1-5 wherein the clustering parameters include a similarity threshold.

11. The invention of claim 10 wherein the similarity threshold is set to 0.95.

12. The invention of claim 11 wherein a first spectrum is clustered into a first spectrum cluster with a second spectrum if the dot product of a first normalized vector representing the first spectrum and a second normalized vector representing the second spectrum is greater than the similarity threshold.

13. The invention of claim 12 wherein a representative spectrum for the first spectrum cluster is chosen based on the higher Xrea value between the first spectrum and the second spectrum.

14. The invention of claims 1-5 wherein the clustering parameters include a retention time tolerance.

15. The invention of claim 14 wherein the retention time tolerance is set to 10 minutes.

16. The inventions of claims 1-5 further comprising generating a PCA of the discriminant spectrum clusters.

17. The inventions of claims 1-5 further comprising:
receiving an unknown input data set comprising a plurality of spectra generated from other biological samples where it is unknown whether the other biological samples have the biological condition;
applying quality control filters to the unknown input data set to remove spectra that do not meet the quality control filters and generate a set of remaining unknown spectra;
clustering the remaining unknown spectra into a second set of spectrum clusters by applying clustering parameters; and
comparing the second set of spectrum clusters to the discriminant spectrum clusters.

18. The invention of claim 17 wherein the comparison of the second set of spectrum clusters to the set of discriminant spectrum clusters is done by computing the Jaccard index of each cluster in the second set of spectrum cluster to each cluster in the set of discriminant spectrum clusters.

19. The invention of claim 18 further comprising identifying whether a biological condition is potentially present in a sample used to generate a spectrum in the second set of spectrum clusters based on the Jaccard index computed of at least one spectrum from the second set of spectrum clusters and at least one spectrum from the set of discriminant clusters.

20. The inventions of claims 1-5 wherein the plurality of spectra in the known input data set further is known to either to have been generated from the biological samples that are known to have or a second biological condition, or known to have been generated from the biological samples that are known not to have a second biological condition.
